(19) United States
(12) Patent Application Publication
(10) Pub. No.: US 2026/0248206 A1
(43) Pub. Date: Aug. 27, 2026

(54) WASHABLE AND REUSABLE ABSORBENT UNDERGARMENT

(71) Applicant: Essity Hygiene and Health Aktiebolag, Göteborg (SE)

(72) Inventors: Philip BLOMSTRÖM, Göteborg (SE); Anna Knös, Göteborg (SE)

(73) Assignee: Essity Hygiene and Health Aktiebolag, Göteborg (SE)

(21) Appl. No.: 18/992,407

(22) PCT Filed: Jul. 13, 2022
(86) PCT No.: PCT/EP2022/069648

§ 371 Date: Jan. 8, 2025

(51) Int. Cl. A41B 9/00; A41B 17/00

(52) U.S. Cl. CPC A41B 9/004; A41B 9/001; A41B 17/00; A41B 2400/60

(57) ABSTRACT

The present disclosure relates to a washable and reusable absorbent undergarment includes an absorbent assembly, including a wearer facing top layer, a moisture barrier layer, and one or more intermediate layers, wherein the absorbent assembly defines a circumferential outer edge and a circumferential margin extending along the circumferential outer edge. The circumferential margin includes one or more sealed margin portions, each sealed margin portion including a fluid seal providing a continuous sealing between the wearer facing top layer and the moisture barrier so as to hinder fluid leakage from an area inside the circumferential margin to the outside of the circumferential outer edge via the sealed margin portion; and the circumferential margin further including one or more unsealed margin portions.

### TECHNICAL FIELD

The disclosure relates to a washable and reusable absorbent undergarment.

### BACKGROUND

An absorbent article, such as an absorbent undergarment, is worn for the purpose of absorbing body fluids such as urine and vaginal fluids. A washable and reusable absorbent undergarment comprises fabrics of woven or knitted materials. Conventionally such a washable and reusable absorbent undergarment comprises an integral absorbent assembly arranged towards the crotch region of the wearer when the undergarment is worn. After use the absorbent undergarment is washed or laundered before reuse.

Reusable absorbent undergarments may be adapted for different volumes of body fluids, from low to moderate or heavy body fluids.

For reusable absorbent undergarments, also those suitable for moderate to heavy flows of body fluids, there is a demand for the undergarment being comfortable to wear as well as providing a good fit and being aesthetically pleasing. As such, there is a desire to provide reusable absorbent undergarments suitable for different flows of body fluids, wherein the required capacity for absorbing body fluids is provided without unnecessary bulk and/or aesthetically disadvantageous effects such as unwanted folds and/or creases.

### SUMMARY

The object of the invention is to meet one or more of the above-mentioned needs, or to provide a useful alternative.

This object is met by a washable and reusable absorbent undergarment according to claim 1.

As such, there is provided a washable and reusable undergarment comprising one or more fabric panels forming a front side and a back side being joined such that the undergarment forms a waist opening and a pair of leg openings with a crotch region extending between the leg openings. A central longitudinal axis of the undergarment extends along the one or more fabric panels of the undergarment in a direction from the back side and towards the front side, and a transversal axis of the undergarment extends along the one or more fabric panels, perpendicular to the central longitudinal axis and dividing the undergarment into the front side and the back side.

In view of the above, the central longitudinal axis will be located centrally as seen over a total transversal width of the undergarment. The transversal axis is to divide the undergarment into the front side and the back side. The location of the transversal axis, i.e. the division between the front side and the back side, may be determined by laying the complete undergarment out with the back of the undergarment towards a supporting surface and the front of the undergarment towards the viewer. The waist edge of the front of the undergarment is to be superposed over the waist edge of the back of the undergarment. When the undergarment is laid flat in this manner, the front side of the undergarment is towards the viewer, the back side of the undergarment is towards the supporting surface, and the transversal axis extends between them, i.e. along a fold between the front side and the back side formed in the crotch region of the undergarment. As such, the transversal axis may be a central transversal axis and located centrally as seen over a total longitudinal length of the undergarment.

The undergarment further comprises an absorbent assembly being arranged in at least part of the crotch region and comprising a wearer facing top layer, a moisture barrier, and one or more intermediate layers being superimposed along a height axis, perpendicular to the longitudinal axis and the transversal axis, between the top layer and the moisture barrier.

The absorbent assembly may be permanently attached to one or more of the fabric panels. For example, the absorbent assembly may be permanently attached to one or more of the fabric panels by one or more mechanical joints such as e.g. seams.

The top layer is arranged so as to face the skin of the user when the undergarment is in use.

The moisture barrier prevents liquid from leaking through the absorbent assembly and may for example be in the form of a liquid-impermeable barrier layer. In other variants however, the moisture barrier may be a liquid-impermeable coating provided e.g. on a garment facing side of an intermediate layer, or optionally on a wearer facing side of a fabric panel underlying the one or more intermediate layers.

The intermediate layer or layers may for example be one or more absorbent layers or an auxiliary layer such as a wicking layer.

Further, the absorbent assembly defines a circumferential outer edge. For example, at least the top layer and the moisture barrier may extend in a plane spanned by the transversal axis and the longitudinal axis so as to terminate essentially at the circumferential outer edge of the absorbent assembly.

Further, a circumferential margin of the absorbent assembly extends along the circumferential outer edge. The circumferential margin constitutes an area in the plane spanned by the longitudinal axis and the transversal axis, and extends along the entire circumferential outer edge of the absorbent assembly. In other words, the circumferential margin forms a closed frame along the circumferential outer edge of the absorbent assembly.

As such, the circumferential margin may extend between the circumferential outer edge of the absorbent assembly and an imaginary inner margin border. The imaginary inner margin border may be uniform in shape to the circumferential outer edge of the absorbent assembly and concentric with the circumferential outer edge.

As proposed herein, the circumferential margin comprises one or more sealed margin portions, each sealed margin portion comprising a fluid seal providing a continuous sealing along the sealed margin portion and between the wearer facing top layer and the moisture barrier so as to hinder fluid leakage from an area inside the circumferential margin to the outside of the circumferential outer edge via the sealed margin portion.

Thus, in each sealed margin portion, the fluid seal provides a continuous sealing as seen in one or more directions along the circumferential direction, and along the sealed margin portion.

As such, each sealed margin portion may be arranged so as to hinder fluid leakage from an area inside the circumferential margin to the outside of the circumferential outer edge via the sealed margin portion.

Further, the circumferential margin comprises one or more unsealed margin portions.

As such, the absorbent assembly is arranged so as not to hinder fluid leakage from an area inside the circumferential margin to the outside of the circumferential outer edge via the one or more unsealed margin portions. Thus, each unsealed margin portion may be free from fluid seals.

As understood from the above, the arrangement of a fluid seal in a sealed margin portion of the circumferential margin is thus efficient to hinder fluid leakage from an area inside the circumferential margin to the outside of the absorbent assembly. As such, the one or more sealed margin portions will be efficient to hinder leakage of fluid from a fluid insult, typically occurring in a fluid insult region of the absorbent assembly located inside the circumferential margin, via the sealed margin portions.

However, the provision of a fluid seal has been found to increase the stiffness of the absorbent assembly at the location of the fluid seal. Thus, although efficient for hindering fluid leakage, any fluid seals may provide the undergarment with an unwanted bulky or less body-forming fit.

By combining one or more sealed margin portions comprising fluid seals with one or more unsealed margin portions, it has been found to be possible to provide adequate hindering of fluid leakage in combination with a better fit. For example, unwanted folds or creases as seen from a front or back side of the undergarment when worn by a user may be reduced or avoided.

As such, the sealed margin portions may be selected to be the margin portions where the absorbent assembly is most prone to leakage. This could for example be where the margin portion is relatively close to an insult region of the absorbent assembly and/or where the margin portion is subject to movement or pressure when in use.

The unsealed margin portions may be selected to be margin portions where the absorbent assembly is less prone to leakage. This could for example be where the margin portion is relatively distant to an insult region of the absorbent assembly. As such, the fluid from a fluid insult can be distributed in a relatively large volume of the absorbent assembly before reaching the margin portions, meaning that little or even no fluid will reach the margin portion when the absorbent article is in use. Also, the unsealed margin portions may be selected taking the comfort and fit of the undergarment into account.

With the term “margin portion” (sealed or unsealed) as used herein, it is to be understood a portion of the circumferential margin as extending along the circumferential direction of the circumferential margin.

With the term “fluid seal” as used herein is consequently meant a fluid seal providing a continuous sealing along the margin portion between the wearer facing top layer and the moisture barrier. As such, the fluid seal provides a continuous sealing in the plane spanned by the longitudinal axis and the transversal axis, and between the wearer facing top layer and the moisture barrier. For example, the extension of the fluid seal along any direction in the plane spanned by the longitudinal axis and the transversal axis may be at least 5 mm, such as at least 10 mm. For example, the extension of the fluid seal along the circumferential edge may be at least 5 mm, such as at least 10 mm.

As such, a sealed margin portion may have a continuous extension along the circumferential edge being at least 5 mm, such as at least 10 mm, or at least 20 mm.

Similarly, an unsealed margin portion may have a continuous extension along the circumferential edge being at least 5 mm, such as at least 10 mm or at least 20 mm.

As such, a washable and reusable undergarment may be provided which is suitable for absorbing a flow of body fluids whilst at the same time providing a satisfactory fit to the body of the user, and having an outer appearance similar to a regular undergarment.

Optionally, the absorbent assembly as such is free from fluid seals outside of the one or more sealed margin portions.

For example, in the one or more sealed margin portions, the fluid seal(s) may be arranged so as to substantially coincide with the circumferential outer edge of the absorbent assembly.

For example, in the one or more sealed margin portions, the fluid seal(s) may be arranged so as to join a circumferential outer edge of the wearer facing top layer with a circumferential outer edge of the moisture barrier. For example, the fluid seal(s) may extend generally along the circumferential outer edges of the top layer and the moisture barrier.

Optionally, in the one or more sealed margin portions, the fluid seal(s) may be arranged to extend along the circumferential outer edge and at a distance from the circumferential outer edge. For example, the fluid seal(s) may be arranged to extend along the circumferential outer edge and at a constant distance from the circumferential outer edge, i.e. the fluid seal(s) have a shape following the contour of the circumferential outer edge.

In another example, the fluid seal(s) may be arranged to extend at a varying distance from the circumferential outer edge. Thus, the shape of the fluid seal(s) may deviate from the contour of the circumferential outer edge.

For example, the distance may be less than 5 mm. For example, the distance may be less than 2 mm, such as less than 1 mm.

Optionally, the one or more fluid seals comprises a fluid sealing joint. A fluid sealing joint is defined as a joint fluid-sealingly joining at least the wearer facing top layer and the moisture barrier to prevent fluid leakage through the fluid sealing joint. For example, each of the one or more fluid seals may comprise a fluid sealing joint.

Optionally, the wearer facing top layer and the moisture barrier are directly joined by at least one of the fluid sealing joints. With “directly” joined is meant herein that the fluid sealing joint joins the garment facing side of the wearer facing top layer and the wearer facing side of the moisture barrier, without any intermediate layers extending in between the top layer and the moisture barrier at the location of the joint.

That the wearer facing top layer and the moisture barrier are directly joined implies that the absorbent assembly may be relatively thin at the location of the fluid sealing joint, thus reducing stiffness and bulkiness of the fluid sealing joint, to the benefit of the undergarment more easily adapting to the wearer's body shape and movements.

Optionally, the wearer facing top layer and the moisture barrier may be indirectly joined. When “indirectly” joined is meant herein that the wearer facing top layer and the moisture barrier are joined by a joint with one or more intermediate layers or other components in between each other at the location of the joint. For example, the garment facing side of the wearer facing top layer may be joined to an intermediate layer, which intermediate layer in turn is attached to the wearer facing side of the moisture barrier.

Optionally, the wearer facing top layer, the moisture barrier, and one or more of the one or more intermediate layers may be joined by at least one of the fluid sealing joints.

Optionally, the wearer facing top layer, the moisture barrier, and all of the one or more intermediate layers are joined by the at least one of the fluid sealing joints.

Optionally, the wearer facing top layer and the moisture barrier may be directly joined by one or more non-fluid sealing joints. For example, at the location of the non-fluid sealing joints, i.e. in the unsealed margin portions, a particularly body-conforming fit may be achieved by the use of non-fluid sealing joints joining only the top layer and the moisture barrier.

For example, in some variants, in the one or more sealed margin portions, the wearer facing top layer and the moisture barrier may be directly joined by one or more fluid sealing joints, and in the one or more unsealed margin portions, the wearer facing top layer and the moisture barrier may be directly joined by one or more non-fluid sealing joints.

In other variants, in the one or more sealed margin portions, the wearer facing top layer, one or more intermediate layers, and the moisture barrier may be joined by one or more fluid sealing joints, and in the one or more unsealed margin portions, the wearer facing top layer and the moisture barrier may be directly joined by one or more non-fluid sealing joints.

The fluid sealing joints may be accomplished in several different ways.

For example, a fluid sealing joint may be accomplished by sealingly joining the layers of the absorbent assembly without addition of any sealing members. For example, a fluid sealing joint may be a fluid sealing adhesive joint, such as a hot melt adhesive joint, or a fluid sealing welded joint, such as a heat welded joint or an ultrasonic welded joint.

In other examples, the one or more fluid seals may comprise a fluid sealing member. For example, each of the one or more fluid seals may comprise a fluid sealing member.

For example, the fluid sealing member may comprise an elastic tape.

For example, the fluid sealing joint may be accomplished by sealingly joining the layers of the absorbent assembly together with a fluid sealing member. For example, the fluid sealing member may comprise an elastic tape being provided with an adhesive providing an adhesive connection to the wearer facing top layer and the moisture barrier.

In another example, the fluid sealing joint may comprise a mechanical joint, such as a seam, in combination with a fluid sealing member.

For example, the fluid sealing joint may comprise a mechanical joint, such as a seam, joining the wearer facing top layer and the moisture barrier, optionally with any intermediate layers, in combination with a fluid sealing member, such as a tape, arranged to extend over the outer edges of the wearer facing top layer, the moisture barrier, and the optional intermediate layers.

In another example, the fluid sealing joint may comprise an adhesive joint, such as an adhesive, joining the wearer facing top layer and the moisture barrier, optionally with any intermediate layers, in combination with a fluid sealing member, such as a tape, arranged to extend over the outer edges of the wearer facing top layer, the moisture barrier, and the optional intermediate layers.

Further, the absorbent assembly may comprise one or more non-fluid sealing joints.

As used herein, a “non-fluid sealing joint” is defined as a joint joining at least the wearer facing top layer and the moisture barrier without preventing fluid leakage through the non-fluid sealing joint.

Optionally, the one or more unsealed margin portions comprises at least one non-fluid sealing joint. For example, each of the one or more unsealed margin portions may comprise at least one non-fluid sealing joint.

Optionally, the non-fluid sealing joint or non-fluid sealing joints may be arranged to fasten the absorbent assembly to at least one out of the one or more of fabric panels.

For example, the non-fluid sealing joint may comprise a mechanical joint such as a seam.

As mentioned in the above, the sealed margin portions may be selected so as to be the margin portions being most prone to leakage from a fluid insult when the article is worn.

As such, optionally, the one or more sealed margin portions may include a pair of sealed side margin portions located opposite to each other and on each side of the longitudinal axis.

Further, each sealed side margin portion may extend longitudinally along at least part of the crotch region of the undergarment.

As may be understood from the above, the sealed side margin portion may per se have an extension along the transversal as well as along the longitudinal axis. That the sealed side margin portion extends longitudinally along at least part of the crotch region of the undergarment implies that the sealed side margin portion has at least an extension along the longitudinal axis in at least part of the crotch region.

Depending on the size and the model of the undergarment, the longitudinal length of the crotch region may vary considerably.

Optionally, the sealed side margin portions may have a longitudinal extension over the entire crotch region of the undergarment. For example, the sealed side margin portions may have a longitudinal extension extending beyond the crotch region of the undergarment towards the front and/or rear of the undergarment. This variant may be particularly useful for undergarment models having a relatively short crotch region, i.e. low leg cut models, such as boxer or shorts models.

Optionally, the sealed side margin portions may have a longitudinal extension over only a part of the crotch region of the undergarment. For example, the sealed side margin portions may have a longitudinal extension over from 20 to 80% of the crotch region, such as over from 25 to 75%. This variant may be particularly useful for undergarment models having a relatively long crotch region, i.e. high leg cut models, such as string or Brazilian models.

Optionally, in some variants, in each sealed side margin portion, at least a part of the circumferential outer edge of the absorbent assembly follows the contour of a respective leg opening of the undergarment.

Optionally, in each sealed side margin portion, at least part of the circumferential outer edge of the absorbent assembly essentially coincides with the contour of a respective leg opening of the undergarment.

For example, when the sealed side margin portions have a longitudinal extension over a part of the crotch region of the undergarment as mentioned in the above, the circumferential outer edge of the absorbent assembly may essentially coincide with the contour of the respective leg opening in the entire part of the crotch region.

Thus, the sealed side margin portions being the margin portions closest to the leg openings of the undergarment may be sealed margin portions.

The sealed side margin portions will thus extend over at least a portion of the longitudinal extension of the leg openings of the undergarment. Optionally, the sealed side margin portions may extend over the entire longitudinal extension of the leg openings of the undergarment.

Optionally, each of the fluid seals in the sealed side margin portions may extend continuously over a longitudinal side sealing length. In other words, the side sealing length is the extension of the sealed side margin portions as seen along the longitudinal axis.

For example, the longitudinal side sealing length may be at least 5 cm, such as at least 7 cm. For example, the longitudinal side sealing length may be at least 10 cm.

For example, the longitudinal side sealing length may be less than 45 cm, such as less than 35 cm.

As such, the longitudinal side sealing length may for example be in the range of from 5 to 45 cm, such as from 10 to 35 cm.

As such, the longitudinal side sealing length may be selected so as to provide for suitable protection against fluid leakage.

For example, the sealed side margin portions may be the only sealed margin portions of the undergarment. I.e. the circumferential margin is free from fluid seals outside of the sealed side margin portions.

Optionally, the one or more unsealed margin portions comprises a front margin portion, wherein the circumferential outer edge of the absorbent assembly faces towards the front of the undergarment. For example, the front margin portion may comprise a non-fluid sealing joint, such as a non-fluid sealing joint joining the absorbent assembly to a fabric panel.

As such, the front margin portion may be free from fluid seals and thus be able to adapt to the user's body shape and movements. Moreover, the shape of the circumferential outer edge of the absorbent assembly in the front margin region may be more freely selected when there is no need to consider the stiffness of any fluid seals.

Optionally, the one or more unsealed margin portions comprises a back margin portion, wherein the circumferential outer edge of the absorbent assembly faces towards the back of the undergarment. For example, the back margin portion may comprise a non-fluid sealing joint, such as a non-fluid sealing joint joining the absorbent assembly to a fabric panel.

As such, the back margin portion may be free from fluid seals and thus be able to better adapt to the user's body shape and movements. Moreover, the shape of the circumferential outer edge of the absorbent assembly in the front margin region may be more freely selected when there is no need to consider the stiffness of any fluid seals.

For example, the one or more unsealed margin portions may comprise a front margin portion and a back margin portion as outlined in the above.

For example, the front margin portion and the back margin portion may interconnect the sealed side margin portions so as to form the complete circumferential margin.

Optionally, the circumferential margin is free from fluid seals outside of the sealed side margin portions.

Optionally, the absorbent assembly is free from fluid seals outside of the sealed side margin portions.

Optionally, the circumferential margin forms a rear margin portion being the portion of the circumferential margin located longitudinally between the rearmost longitudinal end of the sealed side margin portions and a rearmost end of the absorbent assembly.

Optionally, the rear margin portion comprises one or more unsealed margin portions.

An absorbent assembly having a circumferential margin forming a rear margin portion extending longitudinally from the rearmost longitudinal end of the sealed side margin portions towards the back portion of the undergarment enables variants with an extended back side of the absorbent assembly, as may be particularly suitable for undergarments intended for night-time use. By such a rear margin portion comprising one or more unsealed margin portions, better fit and more versatility when it comes to selecting the shape of the rear margin portion may be achieved as outlined in the above.

Optionally, the entire rear margin portion constitutes an unsealed margin portion. As such, the rear margin portion is free from fluid seals.

Optionally, the wearer facing top layer and the moisture barrier layer may extend beyond the one or more intermediate layers in at least a portion of the rear margin portion, such as in the entire rear margin portion. As such, the rear margin portion may be relatively thinner and therefore able to provide a better fit to the user.

Optionally, in the wearer facing top layer and the moisture barrier may extend beyond the one or more intermediate layers in the entire part of the absorbent assembly extending longitudinally along the rear margin portion. In other words, the one or more intermediate layers may extend no further than the sealed side margin portions in a direction towards the rear of the absorbent assembly. As such, the rear portion of the absorbent assembly may be relatively thinner and therefore able to provide a better fit to the user.

The rear margin portion may have a rear margin length along the longitudinal axis.

The Rear Margin Length of the rear margin portion may for example be at least 2 cm, such as at least 4 cm or at least 10 cm. The rear margin length of the rear margin portion may optionally be adapted such that the absorbent assembly extends over the entire longitudinal extension of the back side of the undergarment, i.e. all the way to the waist of the undergarment, such as to a waistband of the undergarment. In other options, the rear margin portion may be adapted such that the absorbent assembly extends over a part of the extension of the back side of the undergarment, such that there is a distance between the waist, e.g. a waistband, of the undergarment and the back end of the absorbent assembly. For example, the rear margin length may less than 40 cm, such as less than 30 cm or less than 25 cm. Optionally, the rear margin length may be from 2 cm to 40 cm, such as from 4 cm to 30 cm.

Optionally, the rear margin portion may comprise one or more non-fluid sealing joints.

Alternatively or in addition, the circumferential margin may form a front margin portion being the portion of the circumferential margin located longitudinally between the frontmost longitudinal end of the sealed side margin portions and a frontmost end of the absorbent assembly.

Optionally, the front margin portion comprises one or more unsealed margin portions.

An absorbent assembly having a circumferential margin forming a front margin portion extending longitudinally from the frontmost longitudinal end of the sealed side margin portions towards the back portion of the undergarment enables variants with an extended back side of the absorbent assembly, as may be particularly suitable for undergarment intended for night-time use. By such a front margin portion comprising one or more unsealed margin portions, better fit and more versatility when it comes to selecting the shape of the front margin portion may be achieved as outlined in the above.

Optionally, the entire front margin portion constitutes an unsealed margin portion. As such, the front margin portion is free from fluid seals.

Optionally, the wearer facing top layer and the moisture barrier layer may extend beyond the one or more intermediate layers in at least a portion of the front margin portion, such as in the entire front margin portion. As such, the front margin portion may be relatively thinner and therefore able to provide a better fit to the user.

Optionally, the wearer facing top layer and the moisture barrier may extend beyond the one or more intermediate layers in the entire part of the absorbent assembly extending longitudinally along the front margin portion. In other words, the one or more intermediate layers may extend no further than the sealed side margin portions in a longitudinal direction towards the front of the absorbent assembly. As such, the front portion of the absorbent assembly may be relatively thinner and therefore able to provide a better fit to the user.

The front margin portion may have a Front Margin Length along the longitudinal axis.

The Front Margin Length of the front margin portion may for example be at least 2 cm, such as at least 4 cm or at least 10 cm. The front margin length of the front margin portion may optionally be adapted such that the absorbent assembly extends over the entire longitudinal extension of the front side of the undergarment, i.e. all the way to the waist of the undergarment, such as to a waistband of the undergarment. In other options, the front margin portion may be adapted such that the absorbent assembly extends over a part of the extension of the front side of the undergarment, such that there is a distance between the waist, e.g. a waistband, of the undergarment and the front end of the absorbent assembly. For example, the front margin length may less than 40 cm, such as less than 30 cm or less than 25 cm. Optionally, the front margin length may be from 2 cm to 40 cm, such as from 4 cm to 30 cm.

Optionally, the front margin portion may comprise one or more non-fluid sealing joints.

For example, in some variants the absorbent assembly may comprise sealed side margin portions and an unsealed rear margin portion with a rear margin length being equal to or greater than the side sealing length. This may be particularly suitable for undergarments intended for night-time use. For example, in some variants the absorbent assembly may comprise sealed side margin portions, an unsealed rear margin portion and an unsealed front margin portion, wherein the rear margin length and/or the front margin length is equal to or greater than the side sealing length.

Optionally, as understood from the above, the circumferential margin may consist of an unsealed front margin portion, an unsealed rear margin portion, and a pair of sealed side margin portions, the sealed side margin portions interconnecting the front margin portion and the rear margin portion.

Optionally, and providing for many alternative variants, such as several of those mentioned in the above, the circumferential margin may consist of a first unsealed margin portion, a second unsealed margin portion, and a pair of sealed side margin portions, the sealed side margin portions interconnecting the first unsealed margin portion and the second unsealed margin portion.

Optionally, the unsealed margin portions have an aggregated longitudinal unsealed length as seen along the longitudinal axis y. The aggregated longitudinal unsealed length is hence the sum of the longitudinal lengths of all transversal portions of the absorbent assembly comprising only unsealed margin portions.

For example, in some variants, the aggregated longitudinal unsealed length may be at least 50% of the side sealing length, such as at least 75% of the side sealing length, or at least equal to the side sealing length.

The shape of the absorbent assembly may be adapted for example to the design of the undergarment, and in view of the need for sufficient absorption and satisfactory fit.

The absorbent assembly may comprise a front edge directed towards the front of the undergarment, a rear edge directed towards the rear of the undergarment, and a pair of side edges, wherein each side edge is at least partly directed towards a respective leg opening and connects the front edge and the rear edge.

Each side edge may be at least partly arranged along the respective leg opening of the undergarment. With “along” is herein meant that the side edge follows the shape of the leg opening and is arranged adjacent or at the respective leg opening.

For example, each side edge may be at least partly arranged at the respective leg opening of the undergarment. This implies that the outer edge of the undergarment towards each leg opening at least partly comprises the side edge of the absorbent assembly.

For example, a minimum transversal assembly crotch width of the absorbent assembly, i.e. a minimum distance between the side edges of the absorbent assembly, may be equal to a minimum transversal undergarment crotch width of the intermediate region of the undergarment.

For example, each side edge of the absorbent assembly may be arranged at the respective leg opening of the undergarment in at least the front intermediate region.

For example, essentially the entire each side edge of the absorbent assembly may be arranged at the respective leg opening of the undergarment.

In other variants, each side edge may be at least partly arranged along the respective leg opening of the undergarment, and with a distance to the respective leg opening. For example, each side edge may in its entirety be arranged along a respecting leg opening, and with a distance to the leg opening.

In some variants, the side edges may comprise concave portions, as seen towards the central longitudinal axis y. For example, the side edges may be generally concave, thus allowing for the shape of the absorbent assembly being relatively wider towards the front and rear of the undergarment, and narrower at a crotch region of the undergarment, as mentioned in the above.

In some variants, the front edge may be convex from the transversal axis. As such, at the location of the central longitudinal axis y, the front edge may protrude towards the front of the undergarment beyond the side edges of the absorbent assembly. This may be beneficial for the fit of the undergarment to the body and contribute to the avoidance of unwanted creases in the fabric panels.

The rear edge of the absorbent assembly may be shaped so as to allow for suitable fit and comfort to a user.

As outlined in the above, the absorbent assembly comprises a wearer facing top layer, a moisture barrier, and one or more intermediate layers being superimposed along a height axis, perpendicular to the central longitudinal axis and the transversal axis, between the top layer and the moisture barrier. As such, the absorbent assembly comprises at least three layers plus the moisture barrier, which in some variants is formed by a moisture barrier layer. Such an absorbent assembly may be particularly suited for a moderate to heavy flow of body fluids.

Optionally, the absorbent assembly comprises a wearer facing top layer, a moisture barrier, and at least two intermediate layers.

Optionally, the absorbent assembly comprises a wearer facing top layer, a moisture barrier, and at least three intermediate layers. As such, the absorbent assembly comprises at least four layers plus the moisture barrier, which in some variants is formed by a moisture barrier layer. Accordingly, the capacity of the absorbent assembly to absorb relatively high flows of body fluid may be further improved, for example to provide an absorbent assembly suited for a heavy flow of body fluids.

For example, the absorbent assembly may comprise no more than nine layers. As such, the absorbent assembly may comprise two, three, four, five, six, seven or eight intermediate layers.

The longitudinal and transversal extension (i.e. the outer shape) of the absorbent assembly per se is defined by the mutual longitudinal and transversal extension of the wearer facing top layer and the moisture barrier.

In some variants, at least one of the at least two intermediate layers may have a longitudinal and transversal extension essentially coinciding with the extension of the absorbent assembly.

In some variants, at least two of the at least two intermediate layers may have a longitudinal and transversal extension essentially coinciding with the extension of the absorbent assembly.

For example, all of the intermediate layers of the absorbent assembly may have a longitudinal and transversal extension essentially coinciding with the extension of the absorbent assembly. I.e. all of the layers of the absorbent assembly have the same longitudinal and transversal extension.

In other variants, the longitudinal and/or transversal extension of at least one out of the three intermediate layers may differ from the longitudinal and/or transversal extension of the absorbent assembly.

For example, the top layer may be a weft knit. In another example, the top layer is of a Jacquard technique. The top layer may for example have a basis weight of 80-200 gsm. Optionally, the top layer may be of polyester, elastane or blends thereof.

Optionally, one or more of the at least two intermediate layers is an absorbent layer. For example, when the absorbent assembly comprises at least three intermediate layers, at least two of the at least three intermediate layers may be absorbent layers.

The basis weight of the absorbent layer(s) may for example be in the range 200-350 gsm. For example, the absorbent layer(s) may be of polyester, polyamide, or a mixture thereof. Optionally, the absorbent layer is of a hydrophilic French terry material.

Optionally, one or more of the at least two intermediate layers may be a wicking layer. For example, the wicking layer may be arranged immediately adjacent the top layer. Optionally, the basis weight of the wicking layer is 180-250 gsm. For example, the wicking layer may be of polyester, polyamid, elastane or a mixture thereof. For example, the wicking layer may be a jersey knit.

The absorbent assembly of the undergarment may be manufactured together with the undergarment as a whole and hence one or more joints joining together the absorbent assembly may also join the absorbent assembly to the one or more fabric panels of the undergarment.

Alternatively, the absorbent assembly of the undergarment may be manufactured as a separate part. As mentioned in the above, the absorbent assembly may, also when manufactured as a separate item, be permanently attached to the undergarment using one or more joints.

Although the option that the absorbent assembly is permanently attached to the undergarment is preferred, the absorbent assembly could instead be releasably attached to the undergarment. For example, the garment-facing side of the absorbent assembly may comprise a fastening member intended for releasably fastening the absorbent assembly to an undergarment. Such a fastening member may be adapted for direct attachment to a fabric panel of an undergarment, or the fastening member may be adapted for attachment to a corresponding fastening member of the undergarment.

Hence, in a second aspect, there is provided a washable and reusable absorbent assembly for an undergarment, the assembly comprising a wearer facing top layer, a moisture barrier, and one or more intermediate layers being superimposed along a height axis, perpendicular to the longitudinal axis and the transversal axis, between the top layer and the moisture barrier, the absorbent assembly defining a circumferential outer edge. A circumferential margin of the absorbent assembly extends along the circumferential outer edge, wherein the circumferential margin comprises one or more sealed margin portions, each sealed margin portion comprising a fluid seal providing a continuous sealing along the margin portion between the wearer facing top layer and the moisture barrier so as to hinder fluid leakage from an area inside the circumferential margin to the outside of the circumferential outer edge via the sealed margin portion; and the circumferential margin further comprising one or more unsealed margin portions.

As such, all features and advantages explained in the above relating to the absorbent assembly of an undergarment may be applied to the absorbent assembly per se, and are encompassed by the present disclosure.

### BRIEF DESCRIPTION OF THE DRAWINGS

The disclosure will be described in greater detail below with reference to the figures shown in the appended drawings, wherein

FIG. 1 shows a front view of a variant of an absorbent undergarment comprising an absorbent assembly;

FIG. 2a shows a view from the wearer-facing side of a first version of the undergarment of FIG. 1 when in a flat laid-out state with the joints between the front side and the back side of the undergarment being removed;

FIG. 2b shows a view from the wearer-facing side of a second version of the undergarment of FIG. 1 when in a flat laid-out state with the joints between the front side and the back side of the undergarment being removed;

FIG. 2c shows a view from the wearer-facing side of a third version of the undergarment of FIG. 1 when in a flat laid-out state with the joints between the front side and the back side of the undergarment being removed;

FIG. 3a shows a front view of another variant of an absorbent undergarment comprising an absorbent assembly;

FIG. 3b shows a view from the wearer-facing side of a first version of the undergarment of FIG. 3 when in a flat laid-out state with the joints between the front side and the back side of the undergarment being removed;

FIG. 3c shows a view from the wearer-facing side of a second version of the undergarment of FIG. 1 when in a flat laid-out state with the joints between the front side and the back side of the undergarment being removed; and

FIGS. 4a to 4e are schematic cross-sectional views of different variants of joints through an absorbent assembly, such as may be used for example in the undergarments of FIGS. 1 to 3c.

### DETAILED DESCRIPTION

Different aspects of the present disclosure will be described more fully hereinafter with reference to the enclosed drawings. The embodiments of the absorbent undergarment disclosed herein can, however be realized in many different forms, such as different sizes and absorption levels, and should not be construed as being limited to the aspects set forth herein. In all figures in the following detailed description, the same reference numerals will be used to indicate the same elements.

As used herein, the term “absorbent undergarment” refers to garments that are worn and intended to be placed against the skin of the wearer to absorb and contain body fluids such as urine and vaginal fluids including menstrual fluid. The undergarment may for example be an underwear, underpants, a panty, or a swimwear of a fabric. The undergarment is intended for adult users. The undergarment may be intended for female use. The undergarment according to the present disclosure is washable, i.e. intended to be laundered or otherwise restored after use for reuse as a sanitary article.

By “washable” it is meant that the absorbent undergarment may be cleaned by laundering. The absorbent undergarment may thus be subjected to an aqueous solution containing detergent without losing its structural features. This aqueous solution may be heated as part of the laundering process, e.g. to 40° C. or 60° C.

The term “fabric” as used in the present disclosure may refer to single or multiple layers of fabrics. The fabric may be knitted or woven.

By “permanently attached”, it is meant that the absorbent assembly is not intended to be separated from the body fabric before, during, or after use. The absorbent assembly is not necessarily directly bonded to the fabric panels, but instead may be attached to the fabric via auxiliary parts such as leg opening edgings or reinforcement patches.

Knitting is a method of constructing a fabric by interlocking a series of loops of one or more yarns. There are two major classes of knitting namely warp and weft knitting.

Weaving is a method or process of interlacing two yarns of similar materials so that they cross each other at right angles to produce woven fabric. The warp yarns run lengthwise in the fabric and the filling threads (weft) or picks, run from side to side.

Jacquard is a system of weaving or knitting that utilizes a versatile pattern mechanism to produce intricate designs by using punch cards controlling the patterns produced. Jacquard knit may be valid for circular, flatbed, warp or weft knit.

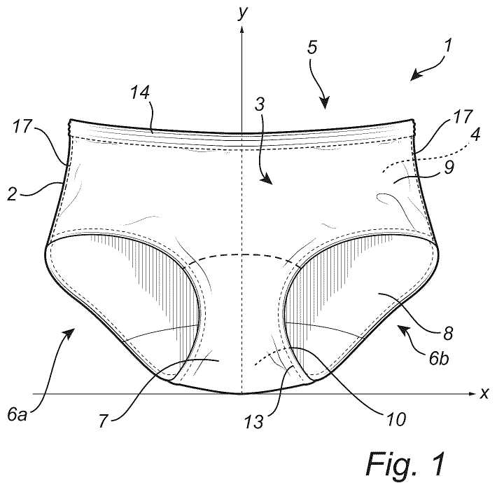

FIG. 1 illustrates as an example of a variant of a washable and reusable absorbent undergarment 1 in the form of a panty. As outlined in the above, the undergarment 1 as proposed herein may however have various designs, such as briefs, boxer, hipster, high waist, string, Brazilian, or other suitable undergarment designs. The undergarment 1 is designed primarily for female use.

The undergarment 1 may comprise one or more fabric panels 2 forming a front side 3 and a back side 4 being joined such that the undergarment 1 forms a waist opening 5 and a pair of leg openings 6a, 6b with a crotch region 7 extending between the leg openings 6a, 6b. A central longitudinal axis y of the undergarment 1 extends along the one or more fabric panels 2 of the undergarment 1 in a direction from the back side 4 and towards the front side 3, and a transversal axis x of the undergarment 1 extends along the one or more fabric panels 2, perpendicular to the central longitudinal axis y and dividing the undergarment 1 into the front side 3 and the back side 4.

In view of the above, the central longitudinal axis y will be located centrally as seen over a total transversal width of the undergarment 1. The transversal axis x is to divide the undergarment 1 into the front side 3 and the back side 4. The location of the transversal axis x, i.e. the division between the front side 3 and the back side 4, may be determined by laying the complete undergarment 1 out with the back of the undergarment 1 towards a supporting surface and the front of the undergarment 1 towards the viewer. The waist edge of the front of the undergarment 1 is to be superposed over the waist edge of the back of the undergarment 1. When the undergarment 1 is laid flat in this manner, the front side 3 of the undergarment 1 is towards the viewer, the back side 4 of the undergarment 1 is towards the supporting surface, and the transversal axis x extends between them, i.e. along a fold between the front side 3 and the back side 4 formed adjacent the crotch region 7 of the undergarment. As such, the front side 3 will be visible from the front of the user when the undergarment 1 is worn, and the back side 4 will be visible from the back of the user when the undergarment 1 is worn. As understood from the above, the transversal axis x may be a central transversal axis of the undergarment 1.

The undergarment of the FIG. 1 example comprises one fabric panel 2, which is cut to form the front side 3 and the back side 4, with the waist opening 5 and the leg openings 6a, 6b. However, in other variants, a plurality of fabric panels may be joined so as to form the front side 3, the back side 4 and the crotch region 7. For example, the undergarment 1 may comprise a front panel, a back panel and a crotch panel. The one or more fabric panels may all comprise the same fabric, giving the undergarment a unitary appearance, or the one or more fabric may comprise different fabrics, for example to provide aesthetically pleasing undergarments comprising e.g. lace fabric.

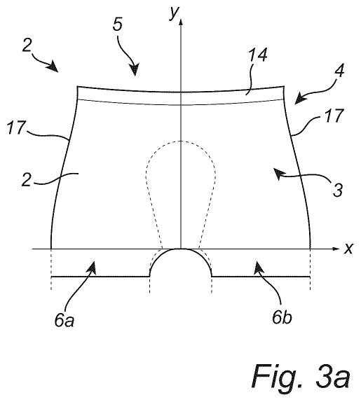

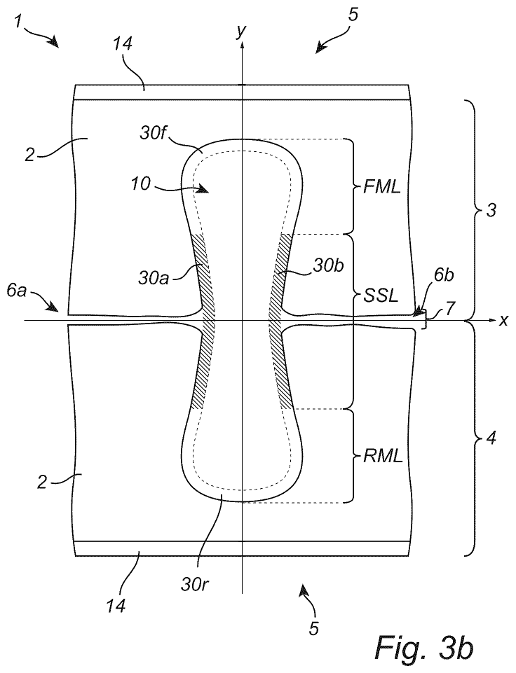

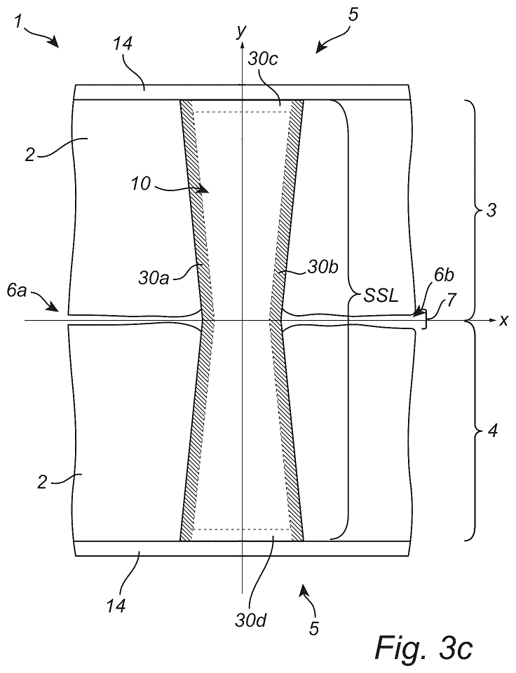

Depending on the cut of the one or more fabric panels 2, different undergarment models may be formed. For example, as in the undergarment model shown in FIG. 1, the leg openings 6a, 6b may have a relatively high cut such as in a bikini model or a Brazilian model. In other examples, such as the undergarment model shown in FIG. 3a, the undergarment 1 may comprise leg portions extending beyond the crotch region 7 in a direction away from the waist opening 5 when the undergarment 1 is worn.

To join the front side 3 and the back side 4 so as to form the waist opening 5 and the leg openings 6a, 6b, a pair of side joints 17 may be provided, as in the illustrated example. The side joints 17 and any other joints joining the one or more fabric panels 2 may be conventional joints in the art, such as seams made by conventional adhesive and/or mechanical bonds such as conventional adhesive or stitching techniques.

As such, the absorbent undergarment 1 may be designed and manufactured in accordance with conventional sewing techniques. For example, the absorbent undergarment may comprise a waist band 14, arranged along the waist opening 5 of the absorbent undergarment 1. For example, the absorbent undergarment 1 may comprise edgings 22a, 22b arranged along the leg openings 6a, 6b.

As mentioned in the above, a central longitudinal axis y of the undergarment 1 is defined along the one or more fabric panels 2 of the undergarment 1 from the back side 4 and towards the front side 3. As such, when an undergarment 1 is seen from the front side 3 as in the example of FIG. 1, the central longitudinal axis y would be directed towards the waist opening 5 of the undergarment 1. However, when the undergarment 1 is seen from the back side 4 (not shown), the central longitudinal axis y would be directed away from the waist opening 5 of the undergarment 1.

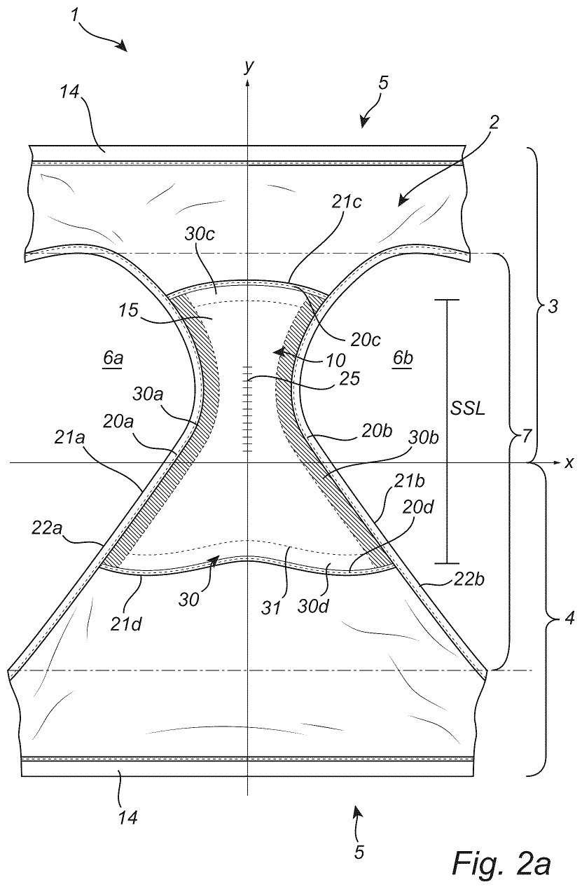

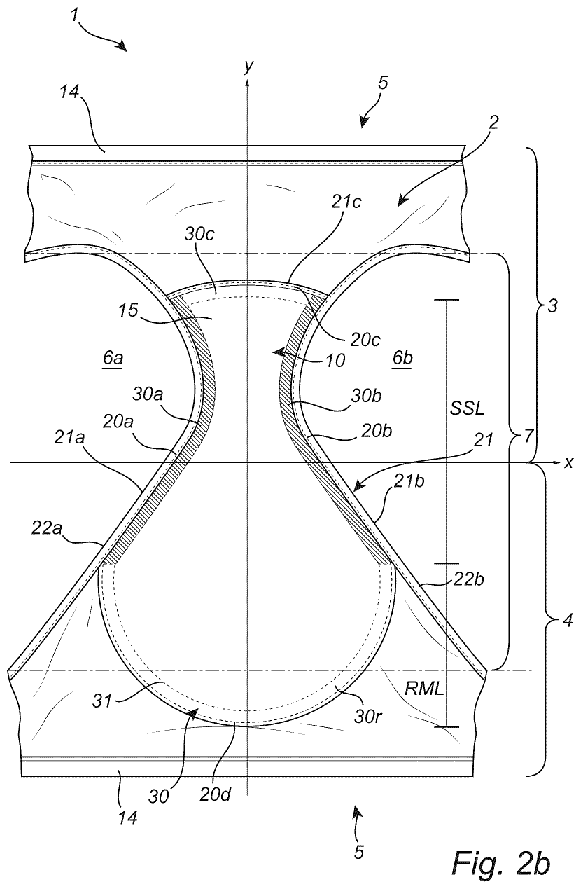

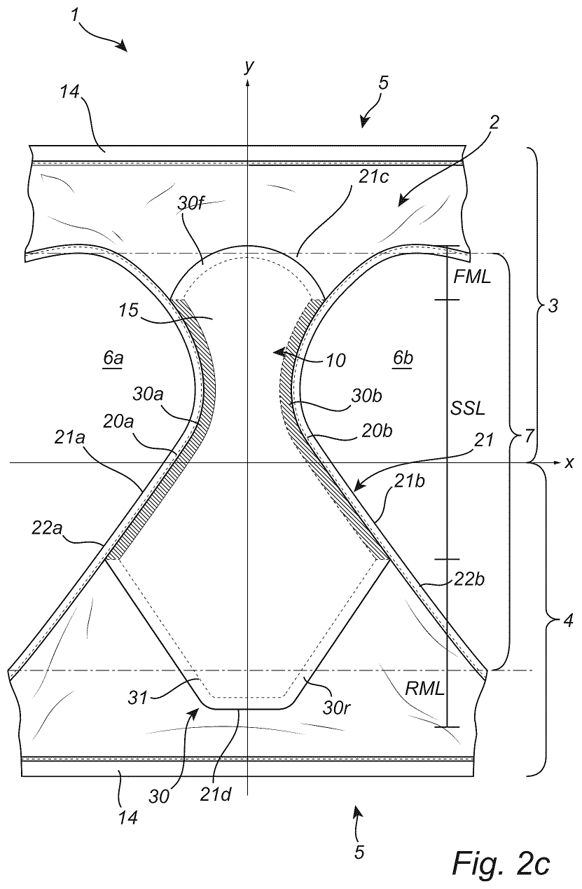

In FIG. 2a, the absorbent undergarment 1 of FIG. 1 is shown in a flat laid-out state, where the side joints 17 of the undergarment 1 are removed. As seen in this laid-out state, the central longitudinal axis y is defined along the one or more fabric panels 2 and in a direction from the rear of the undergarment 1 towards the front of the undergarment 1. In FIG. 2a, the absorbent undergarment 1 is shown from a wearer-facing side of the article 1. As illustrated in FIG. 1, the one or more fabric panels 2 will have an exterior side 9 facing away from the wearer, and an interior side 8 facing in a direction towards the wearer. FIG. 2 thus displays the undergarment 1 in a flat laid-out state as seen from the interior side 8.

As seen in FIG. 2a, the undergarment 1 forms leg openings 6a, 6b. A crotch region 7 is defined as being the portion of the undergarment 1 extending transversely between the leg openings 6a, 6b. As such, the crotch region 7 has a longitudinal extension corresponding to the longitudinal extension of the leg openings 6a, 6b, as indicated in FIG. 2a. It will be understood that depending on the model of the undergarment, i.e. the cut of the leg openings, the longitudinal extension of the crotch region 7 according to this definition may vary considerably.

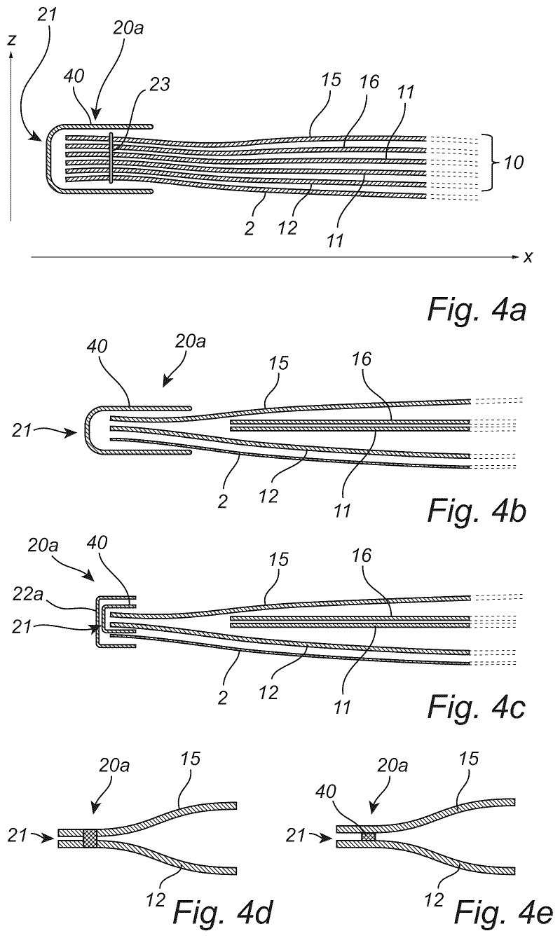

The undergarment 1 further comprises an absorbent assembly 10. As shown in e.g. FIG. 4a, the absorbent assembly 10 comprises a wearer facing top layer 15, a moisture barrier 12, and one or more intermediate layers 11, 16 being superimposed between the top layer 15 and the moisture barrier 12 along a height axis z perpendicular to the central longitudinal axis y and the transversal axis x. The layers 11, 12, 15, 16 of the absorbent assembly 10 will be further described in the below in relation to FIG. 4a.

As seen in FIG. 2a, in the illustrated undergarment 1, the absorbent assembly 10 is located on an interior side 8 of the at least one fabric panel 2. The absorbent assembly 10 is arranged in at least part of the crotch region 7 of the undergarment 1.

Although in some variants the absorbent assembly 10 may extend longitudinally beyond the crotch region 7 in a direction towards the front of the undergarment 1, in other variants, such as the variant illustrated in FIG. 2a, the absorbent assembly 10 extends no further than the crotch region 7 in the longitudinal direction towards the front side 3 of the undergarment 1.

The longitudinal extension of the absorbent assembly 10 towards the rear of the undergarment 1 may depend for example on the design of the undergarment 1. In some variants, such as in the illustrated variant of FIG. 2a, the absorbent assembly 10 may extend no further than the crotch region 7 in the longitudinal direction towards the rear region 4 of the undergarment 1.

As such, and as illustrated in the example of FIG. 2a, the absorbent assembly 10 may be confined to the crotch region 7, wherein the need for absorbance is immediately prevalent.

However, in other variants, the absorbent assembly 10 may extend beyond the crotch region 7 as illustrated e.g. in the variant of FIG. 2b. Further, other options may be available involving the absorbent assembly 10 alternatively or additionally extending beyond the crotch region 7 in a direction towards the front of the undergarment, as illustrated e.g. in the variant of FIG. 2c.

As exemplified by the illustrated undergarment 1, the absorbent assembly 10 forms a circumferential outer edge 21, forming a closed circumference of the absorbent assembly 10.

Further, a circumferential margin 30 is defined as the area in the plane spanned by the longitudinal axis y and the crotch axis x between the circumferential outer edge 21 of the absorbent assembly and an imaginary inner margin border 31. For example, the imaginary inner margin border 31 may be uniform in shape to the circumferential outer edge 21 of the absorbent assembly 10 and concentric with the circumferential outer edge 21. As such, the circumferential margin 30 follows the circumferential outer edge 21.

As proposed herein, the circumferential margin 30 comprises one or more sealed margin portions 30a, 30b, each sealed margin portion 30a, 30b comprising a fluid seal 20a, 20b being arranged so as to hinder fluid leakage from an area inside the imaginary inner margin border 29 to the outside of the circumferential outer edge 21 via the sealed margin portion 30a, 30b. A fluid seal 20a, 20b is in this context a fluid seal providing a continuous sealing along the sealed margin portion 30a, 30b and between the wearer facing top layer 15 and the moisture barrier 12.

(For the purpose of illustration, in the Figures, the sealed margin portions 30a, 30b are shown as hatched portions of the circumferential margin 30. This is merely to illustrate the location of the sealed margin portions 30a, 30b, and does not imply e.g. that the fluid seals 20a, 20b should necessarily extend over the entire areas of the sealed margin portions 30a, 30b.)

As proposed herein, the circumferential margin 30 further comprises one or more unsealed margin portions 30c, 30d. As such, fluid leakage from an area inside the imaginary inner margin border 31 to the outside of the circumferential outer edge 21 via the one or more unsealed margin portions 30c, 30d is not hindered by fluid seals.

However, as will be understood by the example embodiments described herein, the one or more sealed margin portions 30a, 30b, and the one or more unsealed margin portions 30c, 30d may be distributed along the circumferential margin 30 such that sufficient fluid sealing of the absorbent assembly 10 is provided to ensure a well-functioning absorbent undergarment 1, while at the same time providing desirable comfort and fit.

For example, the sealed margin portions may be arranged in pairs such that the sealed margin portions of each pair are located opposite to each other and on each side of the longitudinal axis y. Similarly, the unsealed margin portions may be arranged in pairs such that the unsealed margin portions of each pair are located opposite to each other and on each side of the longitudinal axis y. As such, each pair of unsealed margin portions provide an unsealed longitudinal portion of the absorbent assembly having an unsealed longitudinal length, and each pair of sealed margin portions provide a sealed longitudinal portion of the absorbent assembly having a sealed longitudinal length.

For example, and as illustrated in the variant of FIG. 2a, the one or more sealed margin portions may include a pair of sealed side margin portions 30a, 30b located opposite to each other and on each side of the longitudinal axis y. Each sealed side margin portion 30a, 30b, extends longitudinally along at least a part of the crotch region 7 of the undergarment 1. As mentioned in the above, each sealed side margin portion 30a, 30b thus comprises a fluid seal 20a, 20b providing a continuous sealing between the wearer facing top layer 15 and the moisture barrier 12 so as to hinder fluid leakage from an area inside the circumferential margin 30 to the outside of the circumferential outer edge 21 via the sealed side margin portions 30a, 30b.

When the undergarment 1 is worn, the majority of a fluid insult, such as urine or menses, will contact a central crotch area of the undergarment 1, i.e. an insult region 25. The insult region 25 will be circumscribed ty the imaginary inner margin border 31, and thus the insult region 25 will be circumscribed by the circumferential margin 30.

By provision of the sealed side margin portions 30a, 30b, leakage of the fluid insult towards the leg openings 6a, 6b may be avoided. It has been realized that due to the proximity of the leg openings 6a, 6b to the insult region 25, in combination with the wearer's movements, these areas of the undergarment 1 are otherwise more prone to leakage than other areas.

With the “leg openings” it is herein referred to the portion of the undergarment 1 through which the leg will extend from the crotch region 7 of the undergarment 1. Thus, if the side joints 17 of the undergarment 1 is removed, as exemplified in FIG. 2a, and the crotch region 7 with the absorbent assembly 10 is laid out flat the leg openings 6a, 6b are understood to be the openings as seen in the plane of the transversal axis x and the longitudinal axis y through which the legs of the user will extend along an axis z, perpendicular to the transversal axis x and the longitudinal axis y when the article is worn. Thus, any portions of the undergarments 1 intended to extend along the leg of the wearer when the undergarment 1 are disregarded when determining the extension of the leg openings 6a, 6b. I.e. when the complete undergarment 1 is in a flat state as illustrated in FIG. 1a, any portions of the undergarment extending beyond the location of the transversal axis x in a direction away from the waist opening 5, are to be disregarded (e.g. may be cut off) when determining the extension of the leg openings 6a, 6b.

In some variants, such as the one illustrated in FIG. 2a, in each sealed side margin portion 30a, 30b, at least a part of the circumferential outer edge 21 of the absorbent assembly follows 10 the contour of a respective leg opening 6a, 6b of the undergarment 1. In the variant of FIG. 2a, throughout each sealed side margin portion 30a, 30b, the circumferential outer edge 21 of the absorbent assembly follows the contour of a respective leg opening 6a, 6b.

However, in other variants (not shown), in each sealed side margin portion 30a, 30b, a part of the circumferential outer edge 21 of the absorbent assembly may follow the contour of a respective leg opening 6a, 6b, and another part of the circumferential outer edge 21 may deviate from the contour of a respective leg opening 6a, 6b.

The fluid seals 20a, 20b may, as exemplified in FIG. 2a be arranged so as to substantially coincide with the circumferential outer edge 21 of the absorbent assembly 10.

In other variants, the fluid seals 20a, 20b may be arranged to extend along the circumferential outer edge 21 and at a distance from the circumferential outer edge 21.

In some variants, the distance may be constant, i.e. the one or more fluid seals 20a, 20b may generally follow the contour of the circumferential outer edge 21.

In yet other variants, the distance may vary, i.e. the one or more fluid seals 20a, 20b may have a shape differing more or less from the contour of the circumferential outer edge 21.

In either case, the one or more fluid seals 20a, 20b may advantageously extend relatively close to the circumferential outer edge 21, leaving the majority of the absorbent assembly 10 available for intake and distribution of bodily fluids. Thus, the distance may be for example less than 5 mm. For example, the distance may be less than 2 mm or less than 1 mm.

Optionally, the fluid seals 20a, 20b may comprise one or more fluid sealing joints. As in the illustrated example, each fluid seal 20a, 20b comprises a fluid sealing joint. For example, such a fluid sealing joint may be accomplished by a fluid sealing adhesive joint or a fluid sealing welded joint, such as a heat welded joint or an ultrasonic welded joint.

In some variants, the wearer facing top layer 15 and the moisture barrier 12 may be directly joined by at least one of the fluid sealing joints.

In other variants, the wearer facing top layer 15, the moisture barrier 12, and one or more of the one or more intermediate layers 11, 16 may be joined by at least one of the fluid sealing joints.

In some variants the one or more fluid seals 20a, 20b may comprise a fluid sealing member 40.

For example, the fluid sealing member 40 may be an elastic tape.

In some examples, the fluid sealing member 40 may be an elastic tape being provided with an adhesive, so as to provide a fluid sealing joint comprising a fluid sealing member.

For example, the one or more fluid seals 20a, 20b may comprise a fluid sealing joint comprising an elastic tape being provided with an adhesive for joining the wearer facing top layer 15 and the moisture barrier 12.

Further description of the fluid seals 20a, 20b will be made in the below with reference to FIGS. 4a to 4e.

Each of the fluid seals 20a, 20b in the sealed side margin portions 30a, 30b may extend continuously over a longitudinal side sealing length SSL, as seen along the longitudinal axis y.

To provide sufficient sealing, the longitudinal side sealing length SSL may for example be at least 5 cm, such as at least 10 cm.

The longitudinal side sealing length SSL may for example be less than 45 cm, such as less than 35 cm.

As such, the longitudinal side sealing length SSL may be selected so as to be suitable for providing sufficient hinder to fluid leakage via the sealed side margin portions 30a, 30b.

In addition to the one or more sealed side margin portions 30a, 30b, the circumferential margin 30 of the absorbent assembly 1 comprises one or more unsealed margin portions 30c, 30d. As mentioned in the above, fluid leakage from an area inside the imaginary inner margin border 31 to the outside of the circumferential outer edge 21 via the one or more unsealed margin portions 30c, 30d is not hindered by fluid seals.

Naturally, it is desired to avoid leakage of body fluids from the absorbent assembly 10 as a whole. However, it has now been realized that for some portions of the circumferential margin 30, the risk of leakage occurring is nevertheless small. For example, portions of the circumferential margin 30 that are relatively distanced from the insult region 25 of the undergarment 1 may not be that prone to leakage since the fluid received at the insult region needs to be distributed over a relatively long distance in order to reach such margin portions. Accordingly, portions of the circumferential margin 30 which are deemed not prone to leakage may be selected to be unsealed margin portions.

As such, the unsealed margin portions may be free from fluid seals. Accordingly, for example the shape of the unsealed margin portions need not be restricted by the need to provide fluid seals. Also, the flexibility of the unsealed margin portions is not restricted by the presence of any fluid seals. Accordingly, the unsealed margin portions may be adapted to provide comfort and fit without regard to any problems associated with fluid sealing.

For example, and as illustrated in the variant of FIG. 2a, the circumferential margin 30 may be free from fluid seals outside of the one or more sealed side margin portions 30a, 30b.

For example, and as in FIG. 2a, the absorbent assembly 10 may be free from fluid seals outside of the sealed side margin portions 30a. 30b.

As such, the margin portions of the circumferential margin 30 which are not sealed side margin portions 30a, 30b may be free from fluid seals and may hence be designed without constraints to providing leakage security, to the benefit of fit and comfort.

The unsealed margin portions 30c, 30d may for example comprise non-fluid sealing joints 20c, 20d such as conventional seams or stiches.

For example, and as illustrated in FIG. 2a, the one or more unsealed margin portions may comprise a front margin portion 30c, wherein the circumferential outer edge 21 of the absorbent assembly 10 faces towards the front 3 of the undergarment 1.

For example, and as also illustrated in FIG. 2a, the one or more unsealed margin portions may comprise a back margin portion 30d, wherein the circumferential outer edge 21 of the absorbent assembly 10 faces towards the back 4 of the undergarment 1.

Thus, for example, and as illustrated in FIG. 2a, the circumferential edge 21 may comprise a back edge portion 21d directed towards the back of the undergarment 1, a front edge portion 21c directed towards the front of the undergarment 1, and side edge portions 21a, 21b, directed towards the leg openings 6a, 6b of the undergarment. For example, and as illustrated in the variant of FIG. 2a, the side edge portions 21a, 21b may interconnect the front edge portion 21c and the rear edge portion 21d, so as to form the entire circumferential edge 21. Similarly, the sealed side margin portions 30a, 30b may, as exemplified in FIG. 2a, extend along the side edge portions 21a, 21b and interconnect an unsealed front margin portion 30c extending along the front edge 21c and an unsealed rear margin portion 30d extending along the rear edge 21d.

The shape of the absorbent assembly 10 may be adapted to various absorption needs and to various designs of the undergarment 1, so as to provide sufficient absorption and satisfactory fit.

For example, the side edge portions 21a, 21b may comprise concave portions, as seen towards the central longitudinal axis y. For example, as in the illustrated variant of FIG. 2a, the entire side edge portions 21a, 21b are concave towards the central longitudinal axis y, i.e. curving inwards towards the central longitudinal axis y. Thus, the absorbent assembly 10 may be adapted to fit between the wearer's legs.

The front edge portion 21c of the absorbent assembly may similarly be shaped in view of the need for comfort and fit of the undergarment 1. In some variants, such as in the illustrated undergarment 1, the front edge may be convex from the transversal axis x, i.e. curving away from the transversal axis x. As illustrated in FIG. 2a, at the location of the central longitudinal axis y, the front edge 21c may thus protrude towards the front of the undergarment 1 beyond the extension of the side edge portions 21 a, b of the absorbent assembly 10. This may be beneficial for the fit of the undergarment to the body and contribute to the avoidance of unwanted creases in the fabric panels.

Also, the rear edge portion 21d of the absorbent assembly 10 may be shaped in view of the need for comfort and fit of the undergarment 1. As illustrated in FIG. 2a, the rear edge 21d may for example be concave towards the transversal axis x, i.e. curving towards the central transversal axis x. In other variants, the rear edge 21d may protrude towards the rear of the undergarment 1 beyond the extension of the side edge portions 21a, b of the absorbent assembly 10.

FIG. 2b illustrates another variant of an undergarment 1. Features which are similar to features described in relation to FIG. 2a in the above are denoted with the same reference number. The description of the features made in relation to the variant of FIG. 2a applies equally to the variant of FIG. 2b.

The absorbent assembly 10 illustrated in FIG. 2b comprises a pair of sealed side margin portions 30a, 30b, as described in relation to FIG. 2a.

Further, and as illustrated in FIG. 2b, the circumferential margin 30 of the absorbent assembly 10 may form a rear margin portion 30r extending longitudinally from the rearmost longitudinal end of the sealed side margin portions 30a, 30b towards the back portion 4 of the undergarment 1, to a rearmost end of the absorbent assembly 10. The rear margin portion 30r may comprise one or more unsealed margin portions. For example, and as illustrated in FIG. 2b the one or more unsealed margin portions of the absorbent assembly 10 comprises the rear margin portion 30r, i.e. the absorbent assembly 10 comprises an unsealed rear margin portion 30r. As such, the rear margin portion 30r may be free from fluid seals.

Thus, there is provided an undergarment 1 with a rear margin portion 30r extending rearwardly of the sealed side margin portions 30a, 30b. As such, this may be desired e.g. for forming undergarments 1 particularly suitable for night time use. By ensuring that the rear margin portion 30r is an unsealed margin portion, the outer shape and extension of the absorbent assembly 1 in the rear margin portion 30r need not be restricted by the presence of any fluid seals.

For example, the rear margin portion 30r may have a Rear Margin Length along the longitudinal axis y. The Rear Margin length may for example be at least 2 cm, such as at least 4 cm or at least 10 cm. The Rear margin Length could for example be less than 40 cm, such as less than 30 cm. The Rear Margin Length may be in the range from 2 cm to 40 cm, such as from 4 cm to 30 cm,

Thus, for example, an undergarment 1 may be provided wherein the sealed side margin portion length SSL is between 5 and 35 cm, and the Rear Margin Length is at least 2 cm, such as between 4 and 30 cm.

In some variants, such as illustrated in FIG. 2b, the rearmost end of the absorbent assembly 10 may be arranged at a distance from a rear waist end of the undergarment 1.

For example, the rearmost end of the absorbent assembly 10 may be arranged at a distance of at least 2 cm, such as at least 4 cm or at least 6 cm from the rear waist end of the undergarment 1.

As exemplified in FIG. 2b, the absorbent assembly 10 may for example extend beyond the crotch region 7 of the undergarment 1 in a direction towards the back 4 of the undergarment 1.

FIG. 2c illustrates yet another variant of an undergarment 1. Features which are similar to features described in relation to FIG. 2a or FIG. 2b in the above are denoted with the same reference number, and the description of the features made in relation to the variant of FIG. 2a or FIG. 2b applies equally to the variant of FIG. 2c.

As described with reference to FIG. 2b, in the variant of FIG. 2c, the circumferential margin 30 of the absorbent assembly 10 may form a rear margin portion 30r located in the portion of the absorbent assembly 10 extending longitudinally from the rearmost longitudinal end of the sealed side margin portions 30a, 30b towards the back portion 4 of the undergarment 1, to a rearmost end of the absorbent assembly 10. The rear margin portion may comprise one or more unsealed margin portions 30r. For example, and as illustrated in FIG. 2c the one or more unsealed margin portions of the absorbent assembly 10 comprises the rear margin portion 30r, i.e. the rear margin portion 30r is an unsealed rear margin portion 30r. As such, the rear margin portion 30r may be free from fluid seals.

Further, as in the variant of FIG. 2c, the circumferential margin 30 of the absorbent assembly 10 may form a front margin portion 30f located in the portion of the absorbent assembly 10 extending longitudinally from the frontmost longitudinal end of the sealed side margin portions 30a, 30b to a frontmost end of the absorbent assembly 10. The front margin portion 30f may comprise one or more unsealed margin portions 30f. For example, and as illustrated in FIG. 2c, the one or more unsealed margin portions of the absorbent assembly 10 comprises the front margin portion 30f, i.e. the front margin portion 30r is an unsealed front margin portion 30 r. As such, the front margin portion 30r may be free from fluid seals.

As understood from the examples of FIGS. 2b and 2c, the provision of an unsealed front margin portion 30f and/or an unsealed rear margin portion 30r provides for flexibility when selecting the shape and extension of the front and/or rear margin portions. For example, and as illustrated in the example of FIG. 2b, the contour of the front and/or rear margin portion may comprise rounded shapes, e.g. a bulb shape. In other examples, such as illustrated in FIG. 2c, the contour of the front and/or rear margin portions may comprise straight shapes such as lines and/or corners.

FIG. 3a illustrates yet another variant of an undergarment 1, as shown when the complete undergarment 1 is laid out in a flat state on a support surface. Features which are similar to features described in relation to FIG. 1 and FIGS. 2a to 2c in the above are denoted with the same reference number, and the description of the features made in relation to the variant of FIG. 1 and FIGS. 2a to 2c applies equally to the variant of FIGS. 3a and 3b. For simplicity, FIGS. 3a to 3c are shown with lesser detail than FIGS. 1 to 2c. However, the skilled person will understand that the omitted details such as the location of the sealing and non-sealing joints 20a, 20b, 20c, 20d, may equally be applied and adapted to the variants of FIGS. 3a to 3c.

The undergarment 1 of FIG. 3a differs from the undergarments illustrated in FIGS. 1 and 2a to 2c primarily by that the undergarment 1 is of a model having legs extending beyond the transversal axis x in a direction away from the waist opening 5, when the complete undergarment is laid out as in FIG. 3a. As mentioned in the above, extending leg portions are to be disregarded when determining the leg openings 6a, 6b as defined herein. Thus, In FIGS. 3b and 3c, an undergarment 1 of the model as illustrated in FIG. 3a, is shown with any leg portions extending beyond the transversal axis x in a direction away from the waist opening 5 cut off from the undergarment 1.

Turning to FIG. 3b, which illustrates the undergarment 1 of FIG. 3a in a cut-open state similar to what was described in FIGS. 2a to 2c, it is found that for this type of model, when determining the extension of the leg openings 6a, 6b, these openings may have a relatively short extension along the longitudinal axis y. As such, by definition, the crotch region 7 extending between the leg openings may similarly be relatively short.

The absorbent assembly 10 may, as illustrated in the example of FIG. 3b, extend beyond the crotch region 7 towards the front and the back of the undergarment 1. As described in relation to the other examples, the absorbent assembly 10 may for example comprise a pair of sealed side margin region 30a, 30b, an unsealed front margin region 30f and/or an unsealed rear margin region 30r. For example, and as in the variant of FIG. 3b, the absorbent assembly 10 may comprise an unsealed front margin region 30f and an unsealed rear margin region 30f. The extension and shape of the front and/or rear margin regions may be freely selected, and may, for example, as in FIG. 3b be approximately similar between the front and rear margin regions 30r, 30f. As mentioned in the above, the rearmost end and/or the frontmost end of the absorbent assembly 10 may be arranged at a distance from a waist edge at the waist opening 5 of the undergarment 1.

FIG. 3c illustrates yet another variant of an undergarment 1. The general model of the undergarment in FIG. 3c is similar to the one described in relation to FIGS. 3a and 3b. Features which are similar to features described in relation to FIG. 1 and FIGS. 2a to 2c, or FIGS. 3a to 3b in the above are denoted with the same reference number, and the description of the features made in relation to the variant of FIG. 1 and FIGS. 2a to 2c, or FIGS. 3a to 3b applies equally to the variant of FIG. 3c.

For example, and as illustrated in FIG. 3c, the absorbent assembly 10 may extend over essentially the entire front side 3 and/or rear side 4 of the undergarment 1. As shown in the example of FIG. 3c, the rear end of the absorbent assembly 10 may coincide with a rear waist end of the undergarment 1 as formed by the one or more fabric panels 2. Similarly, and as in the example of FIG. 3c, the front end of the absorbent assembly 10 may coincide with a front waist end of the undergarment 1.

As illustrated in FIG. 3c, the absorbent assembly 10 may comprise sealed side margin portions 30a, 30b, which extend longitudinally over the entire front side 3 and/or back side 4 of the undergarment 1. Thus, the side sealing length SSL corresponds to the total longitudinal extension of the absorbent assembly 10. The absorbent assembly may further comprise an unsealed front margin portion 30c and/or an unsealed rear margin portion 30d. The unsealed front and/or rear margin portions 30c, 30d may coincide with the front waist edge and/or the rear waist edge as formed by the one or more fabric panels, and optionally be provided with a waist band 14.

In other variants, the side sealing length SSL may be less than the total longitudinal extension of the absorbent assembly, as discussed in the above in relation to the variants of FIGS. 1 to 2c. Again, features and explanations made in relation to FIGS. 1 to 2c are applicable also to models such as illustrated in FIGS. 3 to 3c.

The fluid seals and/or the fluid sealing joints of the absorbent assembly 10 may be of any kind as is conventionally known in the art. FIGS. 4a to 4e show different, non-limiting examples of a schematic cross-sectional view of a fluid seal 20a, for example a fluid seal 20a which may be comprised in a sealed side margin portion 30a as illustrated in for example FIG. 2a to 2c, or FIG. 3b to 3c.

The absorbent assembly 10 comprises a wearer facing top layer 15, a moisture barrier 12, and optionally one or more intermediate layers 11, 16 being superimposed between the top layer 15 and the moisture barrier 12 along a height axis z perpendicular to the central longitudinal axis y and the transversal axis x. In the Figures, the layers 11, 12, 15, 16 are illustrated as being spaced from for each other for better visibility. However, in practice the layers will be superimposed over one another, each layer contacting the neighboring upper layer and/or lower layer as seen in the height direction z.

In the variant of FIG. 4a, three intermediate layers 11, 16, in this case two absorbent layers 11 and a wicking layer 16, are shown as an example. Further, the absorbent assembly 10 comprises a liquid-impermeable moisture barrier 12. The liquid-impermeable barrier 12 prevents liquid from leaking through the one or more intermediate layers 11 into the fabric panel 2. The liquid-impermeable barrier 12 may be in the form of a liquid-impermeable barrier layer 12 as illustrated in FIG. 4a. In other variants however, the liquid-impermeable barrier 12 may be a coating on the wearer facing side of a fabric panel 2 or on a garment facing side of an absorbent layer 11. The absorbent assembly 10 further comprises a top layer 15 facing the skin of the user.

In an absorbent assembly 10 comprising one or more intermediate layers 11, 16, all of the layers of the absorbent assembly 10 may in some variants extend throughout the area of the absorbent assembly, i.e. all of the layers extend to the circumferential outer edge 21 of the absorbent assembly 10.

In other variants, one or more of the intermediate layers 11, 16 may extend over less than the complete area of the absorbent assembly 10. In other words, in selected areas of the absorbent assembly 10, the assembly comprises less than all of the layers of the absorbent assembly.

For example, selected areas of the absorbent assembly 10 may comprise only the wearer facing top layer 15 and the moisture barrier 12. This may be advantageous for example for areas of the absorbent assembly 10 where a lesser need for absorption of fluid prevails and/or where better fit and forming of the undergarment 1 to the user's body is required. For example, when the absorbent assembly 10 comprises an unsealed front and/or back margin portion, the longitudinal areas of absorbent assembly corresponding to the unsealed front and/or rear margin portions may be selected areas comprising only the wearer facing top layer and the moisture barrier. As such, the absorbent assembly 10 may comprise the wearer facing top layer 15 and the moisture barrier 12 with no intermediate layers along the front margin length FML and/or the rear margin length RML.

For example, the longitudinal areas of the absorbent assembly 10 corresponding to the longitudinal extension of the sealed side margin portions may comprise the wearer facing top layer 15, the moisture barrier 12 and one or more intermediate layers 11, 16. As such, the absorbent assembly 10 may comprise the wearer facing top layer 15 and the moisture barrier 12 with one or more intermediate layers 11, 16 along the sealed side margin length SSL.

As such, the circumferential margin may, e.g. at the sealed side margin portions, comprise the wearer facing top layer 14, the moisture barrier 12 and one or more intermediate layers 11, 16.

However, in other variants, for one or more circumferential margin portions, the one or more intermediate layers 11, 16 may be arranged so as to be confined inside the circumferential margin 30, as seen in a transversal and/or lateral direction. For example, the one or more intermediate layers 11, 16 may be confined inside the entire circumferential margin.

Where the one or more intermediate layers 11, 16 does not extend into the circumferential margin, joining of the top sheet 15 and the moisture barrier 12 via one or more sealing or non-sealing joints may be facilitated.

Still, it may be desired to join the top sheet 15, the moisture barrier 12 and the one or more intermediate layers 11, 16 with a fluid sealing joint. For example, as in FIG. 4a, a fluid-sealing joint 20a may be arranged in a sealed margin portion comprising a wearer facing top layer 15, a barrier layer 12 and one or more intermediate layers 16, 11. As illustrated herein, a fluid sealing element 40 may be arranged over the outer peripheral edges of the wearer-facing top layer 15, the barrier layer 12, and in this example, the intermediate layers 16, 11. As such, the fluid sealing element 40, which may for example be an elastic tape, forms a continuous sealing between the wearer facing top layer 15 and the moisture barrier 12 so as to hinder fluid leakage in a transversal direction x.

Optionally, and as in FIG. 4a, in addition to the fluid sealing element 40, the fluid sealing joint 21 may comprise a joint 23, joining the layers 15, 12, 16, 11 together inside their peripheral edges. In the illustrated variant, the joint 23 is a mechanical joint, such as a seam. In other variants, the joint 23 could be e.g. an adhesive joint. In the illustrated variant, the joint 23 is arranged so as to be covered by the fluid sealing element 40. In other variants, the joint 23 may be arranged so as to extend through the fluid sealing element 40.

In the example of FIG. 4a, the fluid sealing element 40 extends also over a fabric panel 2 so as to permanently attach the absorbent assembly 10 to the fabric panel. This provides for an efficient attachment of the absorbent assembly 10 to the fabric panel 2 together with the fluid seal 20a. Also, as in the illustrated variant, the joint 23 extends through the fabric panel 2.

However, other variants are possible where the fabric panel 2 is not included in the seal created by the fluid sealing element 40, but the absorbent assembly 10 is permanently attached to one or more fabric panels 2 of the undergarment 1 by other means, for example by a seam.

FIG. 4b differs from FIG. 4a in that there are two intermediate layers 16, 11, in the form of one wicking layer 16 and one absorbent layer 11 in the absorbent assembly 10. As exemplified in FIG. 3b, the intermediate layers 16, 11 need not extend all the way into the circumferential margin 30 of the absorbent assembly 10. Thus, as in the example of FIG. 4b, the fluid sealing element 40 may be arranged over the outer peripheral edges of the wearer facing top layer 15 and the barrier layer 12, where the wearer facing top layer 15 and the barrier layer 12 are in direct contact with each other. As illustrated in FIG. 4b, and as in FIG. 4a, the sealing element 40 may optionally extend also over the outer peripheral edge of a fabric panel 2. The variant of FIG. 4b may be complemented with one or more joints 23, such as adhesive or mechanical joints 23, between the layers, as described in relation to FIG. 4a.

FIG. 4c illustrates an example similar to the example of FIG. 4b, but where the sealing element 40 does not extend over the outer peripheral edge of the fabric panel 2. Instead, the absorbent assembly 10 including the sealing member 40 is attached to the fabric panel 2 by an edging 22a, which extends over the sealing member 40 and the outer peripheral edge of the fabric panel 2. As such, an edging 22a may be used to attach an absorbent assembly 10 as described in any of the examples or illustrations herein to a fabric panel 2. Again, it will be understood that one or more joints 23, such as adhesive or mechanical joints 23 may be applied, e.g. to join the edging 22a and/or in combination with the fluid seal 40 as described in relation to FIGS. 4a and 4b.

FIG. 4d illustrates an example of a variant where the fluid seal 20a comprises a sealing joint, such as a welded joint. In this case, the fluid seal may be formed from melted material. For example, the sealing joint may comprise a hot-melt adhesive. In another example, the sealing joint may comprise melted material from e.g. the layers to be joined together, such as an ultrasound sealing joint or heat sealing joint.

FIG. 4e illustrates an example of a variant where the fluid seal 20a comprises a fluid sealing joint comprising a fluid sealing element 40. For example, the fluid sealing element 40 may be an elastic tape comprising an adhesive.

As understood from the above, the top layer 15 and the moisture barrier 12 may be indirectly joined, i.e. at the joint, the one or more intermediate layers 11, 16 extend between the top layer 15 and the moisture barrier 12 as exemplified in FIG. 4a. For example, the variants illustrated with reference to FIGS. 4b to 4e may be adapted for indirectly joining the top layer 15 and the moisture barrier 12.

Optionally, the top layer 15 and the moisture barrier 12 may be directly joined, i.e. at the joint, the top layer 15 and the moisture barrier 12 are arranged with no intermediate layers 11, 16 between them as exemplified in FIGS. 4b to 4e. However, also the variant illustrated with reference to 4a may be adapted for directly joining the top layer 15 and the moisture barrier 12.

The top layer 15 may be constructed of any suitable fabric, including naturally derived fibers selected from the group consisting of cotton, wool, silk, cellulose, regenerated cellulose, rayon, viscose, modal, lyocell, tencel, bamboo, hemp, flax, ramie, coir or banana. Alternatively, the top layer may be constructed of synthetic fibers selected from the group consisting of polyamide, acrylic, polyester or elastane, such as a mixture of polyester and elastane. Further, the top layer 15 may be constructed of a blend or a mixture of naturally derived and/or synthetic fibers. The materials used for construction of the top layer should be soft and non-irritating to the skin and be readily penetrated by any body fluids. According to the present disclosure, the top layer should be washable. The top layer may comprise between 20% and 100% naturally derived fibers, more preferably between 40% and 100% naturally derived fibers and most preferably between 60% and 100% naturally derived fibers. The top skin-facing layer 15 comprises a water-permeable material thus allowing the body fluids to migrate to the underlying absorbent layer or layers 11. The top layer 15 may have a basis weight of 80-200 gsm.

As exemplified in the variant illustrated in FIGS. 4a to 4e, a wicking layer 16 may be provided underneath the top layer 15. The wicking layer 16 has wicking features to allow the moisture to spread away from the wearer and into the absorbent layer or layers 11. Further, wicking enables an efficient spread of the moisture therefore allowing the moisture to be received in a wider area of the underlying one or more absorbent layers 11. This feature is particularly relevant for users suffering from stress incontinence as spurts of urine may cause the absorbent layer 11 to be saturated rapidly at the point of impact. By spreading the volume of the fluid over a wider receiving area in the absorbent layer 11, the wicking features of the wicking layer 16 increase the overall absorptive capacity of the absorbent layer. The wicking layer 16 may be constructed of any suitable fabric, including naturally derived fibers or mixtures thereof selected from the group consisting of cotton, wool, silk, cellulose, regenerated cellulose, rayon, viscose, modal, lyocell, tencel, bamboo, hemp, flax, ramie, coir or banana. Alternatively, the wicking layer 16 may be constructed of synthetic fibers or mixtures thereof selected from the group consisting of polyamide, acrylic, polyester or elastane. Further, the wicking layer 16 may be constructed of a blend or a mixture of naturally derived and/or synthetic fibers. According to the present disclosure, the wicking layer should be launderable. The wicking layer 16 may comprise between 20% and 100% naturally derived fibers, more preferably between 40% and 100% naturally derived fibers and most preferably between 60% and 100% naturally derived fibers. The basis weight of the wicking layer may be 180-250 gsm.

The absorbent assembly 10 of the absorbent undergarment 1 may contain one or multiple absorbent layers 11, capable of absorbing liquid and releasing the liquid during laundering. Release of the absorbed liquid is beneficial as it enables the absorbent undergarment 1 to be restored for reuse. Reusable absorbent undergarments provide a more sustainable alternative to the commonly used disposable hygiene articles that are not intended to be re-used.

The absorbent layer or layers 11 may comprise any material capable of absorbing fluid, such as woven or nonwoven microfiber or polymer knits, fabric formed from hydrophilic fibers, absorbent fibers or powders. The absorbent layer or layers 11 may be of natural or synthetic fibers as described above for the other ingoing layers but may be of polyester and polyamide and containing odor treatment. The odor treatment may be silver ions, copper ions and zeolites or Polyhexamethylene biguanide, PHMB.

The absorbent layer or layers 11 may also comprise a material having an open cell porous structures such as high loft or synthetic fibers having reservoir properties. The absorbent layer may comprise between 20% and 100% naturally derived fibers, more preferably between 40% and 100% naturally derived fibers and most preferably between 60% and 100% naturally derived fibers. The basis weight of each absorbent layer 11 may be 200-350 gsm. There may be one or more absorbent layers 11 in the absorbent assembly 10. For example, as in the illustrated variant, the absorbent assembly 10 may comprise two absorbent layers 11.

The liquid-impermeable barrier 12 may comprise any suitable material or combinations of material that prevents liquid from migrating from the absorbent assembly 10 to the fabric layer 2. The liquid-impermeable barrier 12 may, as in the illustrated variant, be a liquid-impermeable barrier layer and may comprise a hydrophobic woven or nonwoven material having inherent hydrophobic properties, or that has been treated to become hydrophobic. Examples of hydrophobic materials for treating the barrier layer are polymers such as silicone, polyurethane and combinations thereof. The liquid-impermeable barrier layer 12 may comprise a microporous polymer film, e.g. a polyethylene, PTFE or polyurethane film, or combinations thereof. Laminates of polymer films and nonwoven materials may also be used. Alternatively, the liquid-impermeable barrier 12 may be a coating of a moisture-impermeable material. The coating may be a polymer such as urethane wax provided for example on the surface facing away from the wearer of an absorbent layer 11, or on the surface facing towards the wearer of a fabric panel 2. The liquid-impermeable barrier 12 may be of polyurethane.

The liquid-impermeable barrier 12 is preferably breathable, to allow vapor to escape from the absorbent undergarment 1, while preventing liquid from passing through the fabric layer. Further, the liquid-impermeable barrier 12 may be constructed of an elastic material, thus providing the absorbent assembly 1 with the required flexibility to adapt to the user's anatomy and movements. This improved fit increases the wearer's comfort during use and helps to prevent leakage from migrating through the undergarment's leg openings 6a, 6b.

The fabric of the one or more fabric panels 2 may be constructed of any suitable fabric, including naturally derived fibers and mixtures thereof selected from the group consisting of cotton, wool, silk, cellulose, regenerated cellulose, rayon, viscose, modal, lyocell, tencel, bamboo, hemp, flax, ramie, coir or banana. Alternatively, the fabric may be constructed of synthetic fibers or mixtures thereof selected from the group consisting of polyamide, acrylic, polyester. Further, the fabric may be constructed of a blend or a mixture of naturally derived and/or synthetic fibers. The fibers may be recycled fibers. The fabric may comprise a stretchable fabric, e.g. elastane, so that the absorbent undergarment can provide a firm fit while at the same time adapting to the wearer's movements thus preventing any leakage from migrating through the leg openings and keeping the absorbent assembly in place. The fabric is preferably breathable to allow vapor to escape from the wearer's skin and from the absorbent assembly.

The disclosure may be varied within the scope of the appended claims. For example, the materials and dimensions used for the different layers forming the absorbent assembly may be varied, as indicated above.

1. A washable and reusable undergarment, comprising one or more fabric panels forming a front side and a back side being joined such that the undergarment forms a waist opening and a pair of leg openings with a crotch region extending between the leg openings wherein a central longitudinal axis of the undergarment extends along the one or more fabric panels of the undergarment in a direction from the back side and towards the front side, and a transversal axis of the undergarment extends along the one or more fabric panels, perpendicular to the central longitudinal axis, dividing the undergarment into the front side and said back side;
the undergarment further comprising an absorbent assembly being arranged in at least part of the crotch region and comprising a wearer facing top layer, a moisture barrier, and one or more intermediate layers being superimposed along a height axis (z), perpendicular to the longitudinal axis (y) and the transversal axis, between the top layer and the moisture barrier,
the absorbent assembly defining a circumferential outer edge, wherein
a circumferential margin of the absorbent assembly extends along the circumferential outer edge,
wherein the circumferential margin comprises one or more sealed margin portions, each sealed margin portion comprising a fluid seal providing a continuous sealing along the sealed margin portion and between the wearer facing top layer and the moisture barrier so as to hinder fluid leakage from an area inside the circumferential margin to the outside of the circumferential outer edge via the sealed margin portion; and the circumferential margin further comprising one or more unsealed margin portions.

2. The undergarment according to claim 1, wherein the absorbent assembly is free from fluid seals outside of the one or more sealed margin portions.

3. The undergarment according to claim 1, wherein, in the one or more sealed margin portions, the fluid seal or fluid seals substantially coincide with the circumferential outer edge of the absorbent assembly.

4. The undergarment according to claim 1, wherein, in the one or more sealed margin portions the fluid seals are arranged to extend along the circumferential outer edge and at a distance from the circumferential outer edge.

5. The undergarment according to claim 4, wherein the distance is less than 5 mm.

6. The undergarment according to claim 1, wherein the one or more fluid seals comprises a fluid sealing joint.

7. undergarment according to claim 6, wherein the wearer facing top layer and the moisture barrier are directly joined by at least one of the fluid sealing joints.

8. The undergarment according to claim 6, wherein the wearer facing top layer, the moisture barrier and one or more of the one or more intermediate layers are joined by at least one of the fluid sealing joints.

9. The undergarment according to claim 1, wherein at least one of the one or more fluid seals comprises a fluid sealing member.

10. The undergarment according to claim 9, wherein the fluid sealing member is an elastic tape.

11. The undergarment according to claim 1, wherein at least one of the one or more unsealed margin portions comprises at least one non-fluid sealing joint.

12. The undergarment according to claim 1, wherein at least one of the one or more fluid sealing joints and/or the one or more non-fluid sealing joints comprises a mechanical joint.

13. The undergarment according to claim 1, wherein the one or more unsealed margin portions comprises a front margin portion, wherein the circumferential outer edge of the absorbent assembly faces towards the front of the undergarment.

14. The undergarment according to claim 1, wherein the one or more unsealed margin portions comprises a back margin portion wherein the circumferential outer edge of the absorbent assembly faces towards the back of the undergarment.

15. The undergarment according to claim 1, wherein the one or more sealed margin portions includes a pair of sealed side margin portions located opposite to each other and on each side of the longitudinal axis, wherein optionally each sealed side margin portion extends longitudinally along at least a part of the crotch region of the undergarment.

16. The undergarment according to claim 15, wherein, in each sealed side margin portion, at least a part of the circumferential outer edge of the absorbent assembly follows the contour of a respective leg opening of the undergarment.

17. The undergarment according to claim 15, wherein each of the fluid seals in the sealed side margin portions extends continuously over a longitudinal side sealing length (SSL).

18. The undergarment according to claim 17, wherein the longitudinal side sealing length (SSL) is at least 5.

19. The undergarment according to claim 17, wherein the longitudinal side sealing length (SSL) is less than 45 cm.

20. The undergarment according to claim 15, wherein the circumferential margin free from fluid seals outside of the sealed side margin portions.

21. The undergarment according to claim 15, wherein the absorbent assembly is free from fluid seals outside of the sealed side margin portions.

22. The undergarment according to claim 15, wherein the circumferential margin forms a front margin portion being the part of the circumferential margin located longitudinally between the frontmost longitudinal end of the sealed side margin portions and the frontmost end of the absorbent assembly and/or a rear margin portion being the part of the circumferential margin located between the rearmost longitudinal end of the sealed side margin portions and the rearmost end of the absorbent assembly.

23. The undergarment according to claim 22, wherein the front margin portion and/or the rear margin portion comprises an unsealed margin portion.

24. The undergarment according to claim 22, wherein the wearer facing top layer and/or the moisture barrier layer extend beyond the one or more intermediate layers in at least a portion of the front margin portion and/or rear margin portion.

25. The undergarment according to claim 22, wherein the rear margin portion has a Rear Margin Length (RML) along the longitudinal axis and/or the front margin portion has a Front Margin Length (FML) along the longitudinal axis, and wherein the Rear Margin Length (RML) and/or the Front Margin Length (FML) is at least 2 cm.

26. A washable and reusable absorbent assembly for an undergarment, the assembly comprising a wearer facing top layer, a moisture barrier, and one or more intermediate layers being superimposed along a height axis, perpendicular to the longitudinal axis and the transversal axis, between the top layer and the moisture barrier,
the absorbent assembly defining a circumferential outer edge, wherein
a circumferential margin of the absorbent assembly extends along the circumferential outer edge,
wherein the circumferential margin comprises one or more sealed margin portions, each sealed margin portion comprising a fluid seal providing a continuous sealing along the sealed margin portion between the wearer facing top layer and the moisture barrier so as to hinder fluid leakage from an area inside the circumferential margin to the outside of the circumferential outer edge via the sealed margin portion; and the circumferential margin further comprising one or more unsealed margin portions.

27. (canceled)

28. A washable and reusable absorbent assembly for an undergarment, the assembly comprising a wearer facing top layer, a moisture barrier, and one or more intermediate layers being superimposed along a height axis, perpendicular to the longitudinal axis and the transversal axis, between the top layer and the moisture barrier,
the absorbent assembly defining a circumferential outer edge, wherein
a circumferential margin of the absorbent assembly extends along the circumferential outer edge,
wherein the circumferential margin comprises one or more sealed margin portions, each sealed margin portion comprising a fluid seal providing a continuous sealing along the sealed margin portion between the wearer facing top layer and the moisture barrier so as to hinder fluid leakage from an area inside the circumferential margin to the outside of the circumferential outer edge via the sealed margin portion; and the circumferential margin further comprising one or more unsealed margin portions, wherein the absorbent assembly is free from fluid seals outside of the one or more sealed margin portions.
